(19) United States
(12) Patent Application Publication
(10) Pub. No.: US 2008/0194035 A1
(43) Pub. Date: Aug. 14, 2008

(54) FUEL COMPOSITIONS

(76) Inventors: Richard Edward MALPAS, Ince (GB); Elaine McFarlane, Ince (GB); Keith Reading, Ince (GB); Robert Wilfred Matthews Wardle, Ince (GB)

Correspondence Address: SHELL OIL COMPANY, P O BOX 2463, HOUSTON, TX, 772522463, US

(21) Appl. No.: 11/929,388

(22) Filed: Oct. 30, 2007

(30) Foreign Application Priority Data
Oct. 30, 2006 (EP) 06255560.2

(51) Int. Cl. C10L 1/10; G01N 21/84; G01N 33/22

(52) U.S. Cl. 436/56; 436/60; 436/164

(57) ABSTRACT

A Fischer-Tropsch derived fuel component is provided in a fuel composition to alter the spectroscopic response of the composition to the presence of a spectroscopically active indicator. The Fischer-Tropsch derived component may be used to increase the detectability of the indicator, and/or to increase the resolution of an analytical test subsequently carried out on the composition. Also provided is a method for investigating a property of a fuel composition, by adding a spectroscopically active indicator and detecting a subsequent spectroscopic response in the composition, wherein the composition contains a Fischer-Tropsch derived fuel component. Further provided is a fuel composition containing a spectroscopically active indicator and a Fischer-Tropsch derived fuel component. The spectroscopic response may be a visible response such as a colour change.

### FIELD OF THE INVENTION

The present invention relates to fuel compositions and to their preparation and uses.

### BACKGROUND OF THE INVENTION

Indicators can be added to fuel samples for the purpose of analysis, for example to investigate a property of the fuel such as its origin, age or quality, or the nature or concentration of a component of the fuel or an additive included in it. Many of these indicators provide a visual indication of the outcome of an analysis, which in some cases can be interpreted by eye and is thus convenient for use in the field without recourse to complex analytical equipment or technically skilled operators. Sometimes the visual result is immediate on addition of the indicator to the fuel sample; in others the indicator has to be “developed”, for example by subjecting the sample to specified conditions and/or by adding one or more further reagents, in order to reveal the required result.

Interpretation of such an analysis will typically require the operator to detect either the presence or the absence of a colour change in the fuel sample. In certain cases the nature of the colour change may also provide relevant information—for example, a change to one colour will indicate a first fuel property such as the presence of an additive within a certain concentration range, whereas a change to another colour will indicate a different property such as an additive concentration within a different range. The result may be compared to a reference colour chart in order to interpret the outcome.

It can sometimes be difficult to interpret the results of such analyses, particularly where the relevant colour change is relatively subtle and/or where two or more different outcomes yield similar colours. Interpretation can be even more difficult since many fuels are themselves coloured—typical petroleum-derived diesel fuels, for example, are straw yellow or light brown in colour. This can affect the perceived colour of a sample following analysis. Thus, a colour generated during a test may for example deviate from that shown on the reference colour chart, or two potential indicator colours may appear less readily distinguishable from one another.

An indicator can be incorporated in a fuel prior to its distribution, so as to provide a visual indication of a property of the fuel whether immediately or in response to a subsequent event (for instance a change in a property of the fuel and/or a developing process). Again such indicators (also known as “markers”) typically affect the colour of the fuel. Thus, a fuel may be given a particular colour to indicate for example its origin or quality, whether as a trade mark for the benefit of end users, to allow its subsequent identification or for instance to aid detection of counterfeit or otherwise illegally distributed products.

Again such indicators may be less easy to detect if the fuel itself is already coloured.

### SUMMARY OF THE INVENTION

Accordingly, a method is provided for investigating a property of a fuel composition, the method involving:
(a) adding a spectroscopically active indicator to the composition; and
(b) detecting the presence, absence and/or nature of a spectroscopic response in the composition, on addition of the indicator and/or in response to a subsequent event,
   wherein the fuel composition contains a Fischer-Tropsch derived fuel component.

Further, a method is provided for investigating a property of a fuel composition which already contains a spectroscopically active indicator, the method comprising detecting the presence, absence and/or nature of a spectroscopic response in the composition in response to a subsequent event, wherein the fuel composition contains a Fischer-Tropsch derived fuel component.

### DETAILED DESCRIPTION OF THE INVENTION

It is an aim of the present invention to facilitate the analysis of fuels in such situations, in a manner which can overcome or at least mitigate the above described problems.

Accordingly, a Fischer-Tropsch derived fuel component is provided in a fuel composition for the purpose of altering the spectroscopic response of the composition to the presence of a spectroscopically active indicator.

A “spectroscopically active” indicator is an indicator which is capable of producing a change in the electromagnetic absorption, reflectance, transmission and/or emission spectrum of a fuel composition in which it is present—this change is the “spectroscopic response” of the composition. Thus, a spectroscopic response is a change in the ability of the fuel composition to absorb, reflect, transmit and/or emit electromagnetic radiation, at any one or more frequencies.

The indicator will typically be active in (i.e. produce a change in) the visible and/or ultraviolet (UV) ranges of the electromagnetic spectrum, more typically the visible range.

Thus, a spectroscopic response will typically be or involve a visible response, by which is meant a response which is detectable by the human eye. In other words, a visible response involves a change in the ability of the fuel composition to absorb, reflect, transmit and/or emit electromagnetic radiation in that part of the electromagnetic spectrum between the infrared and ultraviolet regions (referred to in this specification as “visible light”). In particular the response may be or involve a change in the colour of the fuel composition.

A spectroscopic response, and/or an alteration in a spectroscopic response, may be detected by any suitable means, for instance spectroscopy (i.e. by measuring the electromagnetic radiation absorption, reflectance, transmission and/or emission spectrum for the fuel composition at one or more sample frequencies). In an embodiment of the present invention, the response or alteration is detectable by the naked eye.

In general, references to “detecting” a spectroscopic response mean detecting either the presence, the absence and/or the nature of such a response.

The indicator may be present in the fuel composition at any time, whether prior to or more typically subsequent to incorporation of the Fischer-Tropsch derived component. For example, the indicator may be added to the composition (which includes to a sample of the composition) for the purpose of conducting an analysis.

In an embodiment of the present invention, the indicator is generated in situ in the fuel composition, for example as a result of a change in a property of the fuel composition, an event such as the passage of a period of time, and/or a developing process of the type described below which is carried out on an indicator precursor.

In the context of the present invention, a spectroscopically active indicator is any material (either an individual substance or a mixture of two or more substances) which is capable of producing a spectroscopic response, for example a colour change, when included in a fuel composition. The indicator should be capable of signalling, spectroscopically and typically visually, information about a property and/or a change in a property of a fuel composition in which it is present. Such information may be qualitative and/or quantitative with respect to the relevant property. The spectroscopic response or signal will again typically be a visible signal such as a change in the colour of the fuel composition. The signal may occur only in response to a specific property of the fuel composition; if that property is not present it may then be the absence of the signal which conveys information about the relevant property.

The indicator may produce a spectroscopic response (such as a colour change) on addition to the fuel composition—it may, for example, be a dye. It may be capable of producing a spectroscopic response, the nature of which is dependent on a property of the fuel composition. It may instead or in addition be capable of producing a spectroscopic response in response to a subsequent event, such as a change in a property of the fuel composition or a developing process of the type described below. In accordance with the present invention, the Fischer-Tropsch derived fuel component may be used to alter any of these responses.

The indicator may be capable of signalling information about any property of the fuel composition. Examples include the origin of the composition (whether commercial and/or geographical) or of one of its components, the age of the composition, its quality, the nature and/or amount of a component (for example a fuel additive) included in it, and whether and/or to what extent its properties have changed, for example due to oxidative or other degradation processes.

Suitable indicators for use in fuel compositions include acid/base indicators, which produce a spectroscopic response (typically a colour change) in response to a change in pH, the response typically taking place at a precise pH value. Such indicators can be used to detect a range of chemical properties (including constituents) of a fuel composition. Other suitable indicators include fuel dyes, for example of the types described below; fluorescent markers (including those which fluoresce when irradiated, for example with UV light); and biochemical markers or “tags” which can be detected in a fuel sample by means of a specific binding event such as an immunoassay.

Where the spectroscopic response produced by the indicator is a colour change, the indicator may, for example, be capable of changing the fuel composition to a colour selected from yellow, orange, brown, green and blue, preferably from yellow, green and blue. Such colours may be more readily compromised by the natural brown or yellow colour of a typical mineral-derived fuel—for example, a blue or green dye may appear murky brown or black in a typical strongly coloured fuel, and a yellow dye may be difficult to detect in a fuel which is already yellow or brown in colour.

The indicator will suitably be soluble in hydrocarbon-based nonpolar solvents, which are the major constituents of fuel compositions.

An indicator precursor is a material (again either an individual substance or a mixture of two or more substances) which when included in a fuel composition is capable of subsequently generating a spectroscopically active indicator in response, for instance, to a change in a property of the composition, a specific event and/or a developing process. Generation of the indicator is in turn capable of producing a spectroscopic response in the composition.

An indicator developing process is a process which induces a spectroscopic response in a fuel composition containing a spectroscopically active indicator, in particular where there was no previous spectroscopic response in the composition. A developing process thus allows detection of an indicator, where it may not previously have been detectable by spectroscopic means. Such a process may be of known type. It may involve for instance altering a condition of the fuel composition, such as its temperature. It may involve the addition of one or more reagents capable of inducing a chemical change in an indicator or indicator precursor or any other component present in the fuel composition. Typically it will elicit in the overall system a spectroscopic response which was not present prior to the developing process.

An alteration in the spectroscopic response of a fuel composition will alter the way in which the electromagnetic absorption, reflectance, transmission and/or emission spectrum (for example the colour) of the composition changes in response to the indicator. It may alter the response of the composition on addition of the indicator and/or following a subsequent event such as a developing process or a change in a property of the composition.

It may alter the magnitude (e.g. the intensity) of the spectroscopic response of the composition at one or more frequencies, and/or the frequency(ies) at which the spectroscopic response is produced.

It may, for instance, alter the intensity of the colour of the fuel composition, whether before and/or after a colour change produced by the indicator, ideally at least after the change. Suitably, it will increase the intensity of the colour.

Thus, in an embodiment of the present invention, the Fischer-Tropsch derived fuel component is used for the purpose of altering (suitably increasing) the magnitude of a spectroscopic response which the indicator produces in the fuel composition.

Instead or in addition, the Fischer-Tropsch derived fuel component may be used for the purpose of increasing the difference in frequency between two distinct spectroscopic responses which are or can be exhibited by the fuel composition, for instance one before and one after addition of the indicator or as a result of two different fuel composition properties detectable by the indicator.

For example, the Fischer-Tropsch derived fuel component may be used for the purpose of increasing a difference between two colours which the fuel composition is capable of adopting when it contains the indicator, or between the colours of the composition before and after addition of the indicator. Which of the two colours the fuel composition adopts may for instance be influenced by a property of the composition, a specific event and/or an indicator developing process. Again an increase in a colour difference may be an increase in the difference between the electromagnetic absorbance, reflectance, transmission and/or emission spectra of the fuel composition when it adopts each of the two colours.

Thus, in accordance with the present invention, a Fischer-Tropsch derived fuel component may be used, in a fuel composition which either contains or may subsequently contain (or is intended subsequently to contain) a spectroscopically active indicator, for the purpose of heightening the distinction between two spectroscopic responses (such as colours) which the fuel composition is capable of exhibiting in the presence of the indicator.

According to a second aspect of the present invention, a Fischer-Tropsch derived fuel component may be used in a fuel composition for the purpose of increasing the resolution of an analytical test subsequently carried out on the composition, wherein the test involves detecting a spectroscopic response in the composition. The analytical test may be for the purpose of investigating, and suitably determining (whether qualitatively and/or quantitatively), a property of the fuel composition. The test is suitably of the type which involves adding an indicator to the fuel composition and detecting a subsequent spectroscopic response in the composition, for example a visible change such as a change in colour. Again the change may be in response to addition of the indicator, a subsequent change in a property of the composition, a specific event and/or an indicator developing process. The outcome of the test may be indicated by the presence, absence and/or nature (which includes magnitude) of a spectroscopic response in the composition.

The analytical test may be capable of yielding at least two different results, each indicated by a different spectroscopic response (for example, visual signal) in the fuel composition. The present invention can then allow a larger difference, and hence a clearer distinction, between the two results.

For example, the test may be of the type in which a first property of the fuel composition results in a first colour, whereas a second property results in a second, different, colour. The present invention may then be used to increase the difference between the first and second colours, helping the operator to determine which of the two properties is present.

By increasing the intensity of spectroscopic response(s) taking place during such a test, and/or by heightening the distinction between two spectroscopic responses (typically colour changes) which the fuel composition is capable of adopting during the test, the present invention can facilitate detection and/or interpretation of the test result. Results can more easily be distinguished by eye, thus facilitating use of the relevant indicator for instance by less highly trained personnel and in field as well as laboratory tests.

Thus, in general terms, the present invention provides the use of a Fischer-Tropsch derived fuel component, in a fuel composition, for the purpose of facilitating detection of a spectroscopically active indicator present in the composition and/or of a spectroscopic response of the composition to the presence of a spectroscopically active indicator.

In yet another embodiment of the present invention, the Fischer-Tropsch derived fuel component may be used for the purpose of reducing the difference between a spectroscopic response exhibited by the fuel composition during an analytical test—for instance on addition of an indicator and/or in response to a change in property, a specific event and/or an indicator developing process—and a reference response (e.g. a reference spectrum or colour chart) provided for the purpose of interpreting the test results. In other words, the Fischer-Tropsch derived fuel component may be used to generate “truer” spectroscopic responses (e.g. colours or colour changes) when carrying out analytical tests, which can again greatly facilitate interpretation of the test results.

A third aspect of the present invention provides the use of a Fischer-Tropsch derived fuel component, in a fuel composition, for the purpose of reducing the colour of the composition. This in turn can help to reduce the effect of the fuel composition itself on its visible response to an indicator and/or to a subsequent change, event and/or developing process.

Reducing the colour of a fuel composition means reducing the amount of visible light which it absorbs or reflects or emits, and/or increasing the amount of visible light which it transmits. In other words, reducing the colour means making the composition more transparent to visible light (“colourless”).

In the context of the present invention, “use” of a Fischer-Tropsch derived fuel component in a fuel composition means incorporating the component into the composition, typically as a blend (i.e. a physical mixture) with one or more other fuel components. The Fischer-Tropsch derived component will conveniently be incorporated before the composition is introduced into an engine or other system which is to be run on the composition. Instead or in addition the use of a Fischer-Tropsch derived fuel component may involve running a fuel-consuming system, typically an engine such as a diesel engine, on a fuel composition containing the component, typically by introducing the composition into a combustion chamber of an engine.

In general, references to “adding” or “incorporating” a component to a fuel composition may be taken to embrace addition or incorporation at any point during the carrying out of the present invention. Thus, in accordance with the present invention, a fuel composition may be mixed with an indicator and subsequently with a Fischer-Tropsch derived fuel component, or alternatively such a composition may be mixed with a Fischer-Tropsch derived fuel component prior to addition of an indicator.

“Use” of a Fischer-Tropsch derived fuel component in the ways described above may also embrace supplying such a component together with instructions for its use in a fuel composition to achieve one or more of the purposes described above in connection with the first to the third aspects of the present invention, for instance to alter the spectroscopic response of the composition to the presence of a spectroscopically active indicator. The Fischer-Tropsch derived fuel component may be supplied as part of a formulation which is suitable for and/or intended for use as a fuel additive.

In particular, in accordance with a fourth aspect of the present invention, there is provided the use of a Fischer-Tropsch derived fuel component and a spectroscopically active indicator together, in a fuel composition, for one or more of the purposes described above in connection with the first to the third aspects of the present invention. The Fischer-Tropsch derived fuel and the indicator may, for instance, be supplied, and/or added to a fuel composition, in the form of a fuel additive package containing both components, optionally with other fuel additives.

Thus, a fifth aspect of the present invention provides a formulation, suitable for use in a fuel composition, containing both a spectroscopically active indicator and a Fischer-Tropsch derived fuel component.

This aspect of the present invention may be of particular use in analytical tests and test kits. An indicator which is intended to be added to a fuel sample in order to conduct an analytical test may be formulated with a Fischer-Tropsch derived fuel component, so as to alter the spectroscopic response of the sample on addition of the indicator—this in turn can facilitate interpretation of the test results, in the manner described above.

A sixth aspect of the present invention provides an analytical test kit for use in investigating a property of a fuel composition, the kit comprising a Fischer-Tropsch derived fuel component and a spectroscopically active indicator. The Fischer-Tropsch derived component and the indicator may be provided separately, or together as part of a single formulation.

A seventh aspect provides a method for investigating a property of a fuel composition, the method involving adding to the composition (which includes to a sample thereof) a spectroscopically active indicator and a Fischer-Tropsch derived fuel component, and detecting a spectroscopic response in the composition.

It is already known to include Fischer-Tropsch derived fuel components in fuel compositions. Such components are the reaction products of Fischer-Tropsch condensation processes, for example the process known as Shell Middle Distillate Synthesis (van der Burgt et al, “The Shell Middle Distillate Synthesis Process”, paper delivered at the 5th Synfuels Worldwide Symposium, Washington D.C., November 1985; see also the November 1989 publication of the same title from Shell International Petroleum Company Ltd, London, UK). In particular, Fischer-Tropsch derived gas oils are known for inclusion in automotive diesel fuel compositions.

It has now been found that when added to a Fischer-Tropsch derived fuel component, a spectroscopically active indicator can produce a clearer response than when added to a conventional fuel such as a petroleum-derived diesel fuel. For example, there can be a clearer distinction between the different possible outcomes (such as colour changes) of an analytical test involving the indicator. This effect may be at least partially due to the fact that Fischer-Tropsch derived fuels tend to be colourless or substantially so, and may thus be less likely to affect any spectroscopic response produced by the indicator. However, the impact of fuel colour on spectroscopic response is not altogether straightforward, since even in fuels which are relatively pale in colour, indicator responses can still be difficult to distinguish. Thus, it could not necessarily have been predicted that a Fischer-Tropsch derived fuel component could cause a significant improvement in indicator discrimination, as now provided by the present invention.

It is possible, although we do not wish to be bound by this theory, that the chemical constitution of a Fischer-Tropsch derived fuel component may make it less likely to complex with an indicator and hence to modify its spectroscopic properties. Thus, in an embodiment of the present invention, the indicator may for instance be or contain a chemical which is capable of complexing with basic species present in a fuel composition, and/or with another type of species which is present in relatively low levels in, or is absent from, the Fischer-Tropsch derived fuel component.

Moreover, Fischer-Tropsch derived fuels are relatively low in components such as olefins and aromatic species. Such species can affect the electromagnetic absorbing, reflecting, transmitting and/or emitting properties of a fuel. It is possible that lowering the concentrations of such species in a fuel composition can reduce the degree of interference with its spectroscopic response(s) to an indicator present in the composition.

Since it may be desirable to include a Fischer-Tropsch derived component in a fuel composition for other reasons, for example to reduce emissions from a fuel-consuming system (typically an engine) running on the fuel composition, and/or to reduce the level of sulphur, aromatics or other polar components in the composition, the ability to use a Fischer-Tropsch component for the additional purpose of improving the spectroscopic response of the composition to an indicator required in it can provide significant formulation advantages.

The Fischer-Tropsch derived fuel component used in the present invention may be, for example, a Fischer-Tropsch derived naphtha, kerosene or gas oil. In one embodiment it is a gas oil.

By “Fischer-Tropsch derived” is meant that a fuel is, or derives from, a synthesis product of a Fischer-Tropsch condensation process. A Fischer-Tropsch derived fuel may also be referred to as a GTL (Gas-to-Liquid) fuel. The term “non-Fischer-Tropsch derived” may be construed accordingly.

The Fischer-Tropsch reaction converts carbon monoxide and hydrogen into longer chain, usually paraffinic, hydrocarbons:

n(CO+2H₂)=(—CH₂—)ₙ+nH₂O+heat,

in the presence of an appropriate catalyst and typically at elevated temperatures (e.g. 125 to 300° C., preferably 175 to 250° C.) and/or pressures (e.g. 5 to 100 bar, preferably 12 to 50 bar). Hydrogen:carbon monoxide ratios other than 2:1 may be employed if desired.

The carbon monoxide and hydrogen may themselves be derived from organic or inorganic, natural or synthetic sources, typically either from natural gas or from organically derived methane. The gases which are converted into liquid fuel components using such processes can in general include natural gas (methane), LPG (e.g. propane or butane), “condensates” such as ethane, synthesis gas (CO/hydrogen) and gaseous products derived from coal, biomass and other hydrocarbons.

Gas oil, naphtha and kerosene products may be obtained directly from the Fischer-Tropsch reaction, or indirectly for instance by fractionation of Fischer-Tropsch synthesis products or from hydrotreated Fischer-Tropsch synthesis products. Hydrotreatment can involve hydrocracking to adjust the boiling range (see, e.g., GB-B-2077289 and EP-A-0147873) and/or hydroisomerisation which can improve cold flow properties by increasing the proportion of branched paraffins. EP-A-0583836 describes a two step hydrotreatment process in which a Fischer-Tropsch synthesis product is firstly subjected to hydroconversion under conditions such that it undergoes substantially no isomerisation or hydrocracking (this hydrogenates the olefinic and oxygen-containing components), and then at least part of the resultant product is hydroconverted under conditions such that hydrocracking and isomerisation occur to yield a substantially paraffinic hydrocarbon fuel. The desired gas oil fraction(s) may subsequently be isolated for instance by distillation.

Other post-synthesis treatments, such as polymerisation, alkylation, distillation, cracking-decarboxylation, isomerisation and hydroreforming, may be employed to modify the properties of Fischer-Tropsch condensation products, as described for instance in U.S. Pat. No. 4,125,566 and U.S. Pat. No. 4,478,955.

Typical catalysts for the Fischer-Tropsch synthesis of paraffinic hydrocarbons comprise, as the catalytically active component, a metal from Group VIII of the periodic table, in particular ruthenium, iron, cobalt or nickel. Suitable such catalysts are described for instance in EP-A-0583836 (pages 3 and 4).

An example of a Fischer-Tropsch based process is the SMDS (Shell Middle Distillate Synthesis) described by van der Burgt et al in “The Shell Middle Distillate Synthesis Process”, supra. This process (also sometimes referred to as the Shell “Gas-To-Liquids” or “GTL” technology) produces middle distillate range products by conversion of a natural gas (primarily methane) derived synthesis gas into a heavy long chain hydrocarbon (paraffin) wax which can then be hydroconverted and fractionated to produce liquid transport fuels such as the gas oils useable in automotive diesel fuel compositions. A version of the SMDS process, utilising a fixed bed reactor for the catalytic conversion step, is currently in use in Bintulu, Malaysia and its gas oil products have been blended with petroleum derived gas oils in commercially available automotive fuels.

Gas oils, naphthas and kerosenes prepared by the SMDS process are commercially available, for instance, from Shell companies. Further examples of Fischer-Tropsch derived gas oils are described in EP-A-0583836, EP-A-1101813, WO-A-97/14768, WO-A-97/14769, WO-A-00/20534, WO-A-00/20535, WO-A-00/11116, WO-A-00/11117, WO-A-01/83406, WO-A-01/83641, WO-A-01/83647, WO-A-01/83648 and U.S. Pat. No. 6,204,426.

By virtue of the Fischer-Tropsch process, a Fischer-Tropsch derived fuel has essentially no, or undetectable levels of, sulphur and nitrogen. Compounds containing these heteroatoms tend to act as poisons for Fischer-Tropsch catalysts and are therefore removed from the synthesis gas feed. Further, the Fischer-Tropsch process as usually operated produces no or virtually no aromatic components: the aromatics content of a Fischer-Tropsch derived fuel, suitably determined by ASTM D4629, will typically be below 1% w/w, preferably below 0.5% w/w and more preferably below 0.1% w/w.

Generally speaking, Fischer-Tropsch derived fuels have relatively low levels of polar components, in particular polar surfactants, for instance compared to petroleum derived fuels. Such polar components may include for example oxygenates, and sulphur- and nitrogen-containing compounds. A low level of sulphur in a Fischer-Tropsch derived fuel is generally indicative of low levels of both oxygenates and nitrogen-containing compounds, since all are removed by the same treatment processes.

Where a Fischer-Tropsch derived fuel component is a naphtha fuel, it will be a liquid hydrocarbon distillate fuel with a final boiling point of typically up to 220° C. or preferably of 180° C. or less. Its initial boiling point is preferably higher than 25° C., more preferably higher than 35° C. Its components (or the majority, for instance 95% w/w or greater, thereof) are typically hydrocarbons having 5 or more carbon atoms; they are usually paraffinic.

In the context of the present invention, a Fischer-Tropsch derived naphtha fuel preferably has a density of from 0.67 to 0.73 g/cm³ at 15° C. and/or a sulphur content of 5 mg/kg or less, preferably 2 mg/kg or less. It preferably contains 95% w/w or greater of iso- and normal paraffins, preferably from 20 to 98% w/w or greater of normal paraffins. It is preferably the product of a SMDS process, preferred features of which may be as described below in connection with Fischer-Tropsch derived gas oils.

A Fischer-Tropsch derived kerosene fuel is a liquid hydrocarbon middle distillate fuel with a distillation range suitably from 140 to 260° C., preferably from 145 to 255° C., more preferably from 150 to 250° C. or from 150 to 210° C. It will have a final boiling point of typically from 190 to 260° C., for instance from 190 to 210° C. for a typical “narrow-cut” kerosene fraction or from 240 to 260° C. for a typical “full-cut” fraction. Its initial boiling point is preferably from 140 to 160° C., more preferably from 145 to 160° C.

A Fischer-Tropsch derived kerosene fuel preferably has a density of from 0.730 to 0.760 g/cm³ at 15° C.—for instance from 0.730 to 0.745 g/cm³ for a narrow-cut fraction and from 0.735 to 0.760 g/cm³ for a full-cut fraction. It preferably has a sulphur content of 5 mg/kg or less. It may have a cetane number of from 63 to 75, for example from 65 to 69 for a narrow-cut fraction or from 68 to 73 for a full-cut fraction. It is preferably the product of a SMDS process, preferred features of which may be as described below in connection with Fischer-Tropsch derived gas oils.

A Fischer-Tropsch derived gas oil should be suitable for use as a diesel fuel, ideally as an automotive diesel fuel; its components (or the majority, for instance 95% w/w or greater, thereof) should therefore have boiling points within the typical diesel fuel (“gas oil”) range, i.e. from 150 to 400° C. or from 170 to 370° C. It will suitably have a 90% w/w distillation temperature of from 300 to 370° C.

A Fischer-Tropsch derived gas oil will typically have a density from 0.76 to 0.79 g/cm³ at 15° C.; a cetane number (ASTM D613) greater than 70, suitably from 74 to 85; a kinematic viscosity (ASTM D445) from 2 to 4.5, preferably from 2.5 to 4.0, more preferably from 2.9 to 3.7, mm²/s at 40° C.; and a sulphur content (ASTM D2622) of 5 mg/kg or less, preferably 2 mg/kg or less.

Preferably, a Fischer-Tropsch derived fuel component used in the present invention is a product prepared by a Fischer-Tropsch methane condensation reaction using a hydrogen/carbon monoxide ratio of less than 2.5, preferably less than 1.75, more preferably from 0.4 to 1.5, and ideally using a cobalt containing catalyst. Suitably, it will have been obtained from a hydrocracked Fischer-Tropsch synthesis product (for instance as described in GB-B-2077289 and/or EP-A-0147873), or more preferably a product from a two-stage hydroconversion process such as that described in EP-A-0583836 (see above). In the latter case, preferred features of the hydroconversion process may be as disclosed at pages 4 to 6, and in the examples, of EP-A-0583836.

Suitably, in accordance with the present invention, a Fischer-Tropsch derived fuel component will consist of at least 70% w/w, preferably at least 80% w/w, more preferably at least 90% w/w, most preferably at least 95 or 98 or even 99% w/w, of paraffinic components, preferably iso- and normal paraffins. The weight ratio of iso-paraffins to normal paraffins will suitably be greater than 0.3 and may be up to 12; suitably it is from 2 to 6. The actual value for this ratio will be determined, in part, by the hydroconversion process used to prepare the fuel from the Fischer-Tropsch synthesis product.

The olefin content of the Fischer-Tropsch derived fuel component is suitably 0.5% w/w or lower. Its aromatics content is suitably 0.5% w/w or lower.

According to the present invention, a mixture of two or more Fischer-Tropsch derived fuel components may be used in a fuel composition.

The Fischer-Tropsch derived fuel component may be added to the fuel composition for one or more other purposes in addition to the desire to alter the spectroscopic response of the composition to an indicator, for instance to reduce emissions (regulated and/or carbon dioxide) from a fuel-consuming system running on the fuel composition, or to reduce the level of sulphur and/or aromatics and/or other polar components in the composition. Thus, the present invention can be used to optimise the properties and performance of a fuel composition in a number of ways, and can therefore provide additional flexibility in fuel formulation.

The concentration of the Fischer-Tropsch derived component in the fuel composition, after carrying out the present invention, will preferably be 50% v/v or greater, more preferably 60% v/v or greater, yet more preferably 70 or 80 or 85 or 90 or 95% v/v or greater. Its concentration may be up to 100% (i.e. the composition may consist entirely of the Fischer-Tropsch derived fuel), or up to 99.99% v/v, such as up to 99.8 or 99.5 or 99 or 98% v/v. Most preferably, its concentration is from 60 to 100% v/v, such as from 70 or 80 to 100% v/v.

The remainder of the fuel composition will typically contain a major proportion (by which is meant typically 80% v/v or greater, more suitably 90 or 95% v/v or greater, most preferably 98 or 99 or 99.5 or 99.8% v/v or greater) of, or consist essentially or entirely of, a base fuel such as a distillate hydrocarbon base fuel, optionally together with one or more additional components such as fuel additives. Such a base fuel may contain one or more non-Fischer-Tropsch derived (for example petroleum derived) fuel components.

A base fuel may, for example, be a naphtha, kerosene or diesel fuel, preferably a diesel fuel. It may be a fuel which, at least prior to incorporation of the Fischer-Tropsch derived fuel component, is coloured (i.e. absorbs and/or reflects one or more frequencies of visible light). It may for instance be yellow or brown in colour, as are many petroleum derived diesel base fuels.

Thus, a fuel composition prepared according to the present invention may be, for example, a naphtha, kerosene or diesel fuel composition, preferably kerosene or diesel, more preferably diesel. It may in particular be a middle distillate fuel composition, for example a heating oil, an industrial gas oil, an on- or off-road automotive diesel fuel, a railroad diesel fuel, a distillate marine fuel, a diesel fuel for use in mining applications or a kerosene fuel such as an aviation fuel or heating kerosene. Preferably, the fuel composition is for use in an engine such as an automotive engine or an aircraft engine. More preferably, it is for use in an internal combustion engine; yet more preferably, it is an automotive fuel composition, still more preferably a diesel fuel composition which is suitable for use in a compression ignition engine. The present invention may generally be of use for any fuel composition which is intended for, and/or adapted for, use in a compression ignition engine.

A naphtha base fuel will typically boil in the range from 25 to 175° C. A kerosene base fuel will typically boil in the range from 150 to 275° C. A diesel base fuel will typically boil in the range from 150 to 400° C.

The base fuel may in particular be a middle distillate base fuel, in particular a diesel base fuel, and in this case it may itself comprise a mixture of middle distillate fuel components (components typically produced by distillation or vacuum distillation of crude oil), or of fuel components which together form a middle distillate blend. Middle distillate fuel components or blends will typically have boiling points within the usual middle distillate range of 125 to 550° C. or 150 to 400° C.

A diesel base fuel may be an automotive gas oil (AGO). Typical diesel fuel components comprise liquid hydrocarbon middle distillate fuel oils, for instance petroleum derived gas oils. Such base fuel components may be organically or synthetically derived. They will typically have boiling points within the usual diesel range of 125 or 150 to 400 or 550° C., depending on grade and use. They will typically have densities from 0.75 to 1.0 g/cm³, preferably from 0.8 to 0.9 or 0.86 g/cm³, at 15° C. (IP 365) and measured cetane numbers (ASTM D613) of from 35 to 80, more preferably from 40 to 75 or 70. Their initial boiling points will suitably be in the range 150 to 230° C. and their final boiling points in the range 290 to 400° C. Their kinematic viscosity at 40° C. (ASTM D445) might suitably be from 1.5 to 4.5 mm²/s.

Such fuels are generally suitable for use in compression ignition (diesel) internal combustion engines, of either the indirect or direct injection type.

A petroleum derived gas oil may be obtained by refining and optionally (hydro)processing a crude petroleum source. It may be a single gas oil stream obtained from such a refinery process or a blend of several gas oil fractions obtained in the refinery process via different processing routes. Examples of such gas oil fractions are straight run gas oil, vacuum gas oil, gas oil as obtained in a thermal cracking process, light and heavy cycle oils as obtained in a fluid catalytic cracking unit and gas oil as obtained from a hydrocracker unit. Optionally, a petroleum derived gas oil may comprise some petroleum derived kerosene fraction.

Such gas oils may be processed in a hydrodesulphurisation (HDS) unit so as to reduce their sulphur content to a level suitable for inclusion in an automotive fuel composition. This also tends to reduce the content of other polar species such as oxygen- or nitrogen-containing species.

A base fuel may be or contain a so-called “biofuel” component such as a vegetable oil or vegetable oil derivative (e.g. a fatty acid ester, in particular a fatty acid methyl ester) or another oxygenate such as an acid, ketone or ester. Such components need not necessarily be bio-derived.

The base fuel preferably has a low sulphur content, for example at most 1000 mg/kg. More preferably, it will have a low or ultra low sulphur content, for instance at most 500 mg/kg, preferably no more than 350 mg/kg, most preferably no more than 100 or 50 or 10 or even 5 mg/kg, of sulphur. It may be a so-called “zero-sulphur” fuel. Ideally a fuel composition which results from carrying out the present invention will also have a sulphur content falling within these limits.

According to the present invention, the fuel composition may contain one or more additives in addition to the Fischer-Tropsch derived fuel component. Many such additives are known and readily available. The total additive content in the fuel composition may suitably be from 50 to 10000 mg/kg, preferably below 5000 mg/kg.

According to an eighth aspect of the present invention, there is provided a method for investigating a property of a fuel composition, the method involving:
(a) adding a spectroscopically active indicator to the composition (which includes to a sample thereof); and
(b) detecting the presence, absence and/or nature of a spectroscopic response in the composition, on addition of the indicator and/or in response to a subsequent event,
   wherein the fuel composition contains a Fischer-Tropsch derived fuel component.

This aspect of the present invention also encompasses a method for investigating a property of a fuel composition which already contains a spectroscopically active indicator, the method involving detecting the presence, absence and/or nature of a spectroscopic response in the composition in response to a subsequent event, wherein the fuel composition contains a Fischer-Tropsch derived fuel component. The Fischer-Tropsch component may be added to the composition after the indicator, for instance in readiness for the detection step.

The spectroscopic response may in particular be or involve a colour change.

The Fischer-Tropsch derived fuel component is suitably included in the composition for one or more of the purposes described above in connection with the first to the fifth aspects of the present invention.

A method according to the eighth aspect of the present invention may, for example, be used for quality control or assurance purposes, for market research, for testing compliance with regulatory requirements or other relevant specifications, for detecting counterfeit products or for tracking the distribution or use of a fuel composition.

In addition to their use for analytical purposes, it is also known to incorporate spectroscopically active indicators in fuel compositions, for example at the refinery or at the tanker refuelling depot or gantry, in order to provide a detectable indication of a property of a fuel composition, whether immediately or in response to a subsequent event such as a change in a property of the composition and/or an indicator developing process. Again such indicators (also known as “markers”) typically affect the colour of the composition. In this way, a fuel may be given a particular colour to indicate, for example, its origin or quality, whether as a trade mark for the benefit of end users, to aid its subsequent identification or to aid detection of counterfeit or otherwise illegally traded products or of illegally dumped fuel products. In some countries, for example, it is a legal requirement to dye fuels which fail to meet a particular quality specification, or which are for example low- as opposed to high-tax fuels; again, this assists in quality control and in ensuring compliance with the relevant laws and regulations.

One example is the lower-taxed so-called “red diesel” which is available to certain users in the UK as an alternative to more heavily taxed automotive diesel fuels. Mandatory inclusion of the red dye helps prevent its sale and use in inappropriate contexts and assists the authorities in policing the relevant laws.

Another example is the mandatory incorporation of Solvent Yellow 124 into tax-rebated fuels such as heating oils marketed within the EU. This dye changes from yellow to red at acidic pHs, and can hence be detected, even at relatively low concentrations, by acidification of a sample such as with dilute hydrochloric acid. This enables, for example, automotive diesel fuels to be checked for illicit inclusion of lower-taxed fuels.

In the USA, high sulphur content fuels intended for off-road use have to be marked with a red dye, for instance Solvent Red 26 or Solvent Red 164. Tax-exempt diesel fuels also have to be marked in the same way. If a red-dyed fuel is detected in the fuel system of a road vehicle, this can incur substantial penalties.

Again indicators of this type may be less readily detectable, and hence less effective, if the fuel composition itself is already coloured. The present invention may be used to improve their detectability in the same way as it can for any other spectroscopically active indicators.

Thus, in accordance with a ninth aspect of the present invention, there is provided the use of a Fischer-Tropsch derived fuel component, in a fuel composition, for the purpose of improving the detectability of a spectroscopically active indicator in the composition. The indicator may be added to the composition after the Fischer-Tropsch derived fuel component, for instance in order to conduct an analytical test.

Detectability of an indicator will typically be its visibility, for example to the human eye. Improving detectability may embrace increasing the magnitude (e.g. intensity) of a spectroscopic response produced by the indicator, and/or increasing the difference between two or more spectroscopic responses of the fuel composition, at least one of which is produced by the indicator.

According to a tenth aspect of the present invention, there is provided a fuel composition containing a spectroscopically active indicator and a Fischer-Tropsch derived fuel component.

Such a fuel composition may, for example, be a diesel or kerosene fuel composition, more preferably a diesel fuel composition, such as an automotive diesel fuel. It may in general terms be a fuel composition which is intended and/or adapted and/or suitable for use in a compression ignition engine.

Again, the Fischer-Tropsch derived fuel component may be used to help strengthen and/or clarify a spectroscopic response (in particular a visible signal such as a colour or colour change) produced by the indicator.

The indicator will typically “mark” the fuel composition by imparting a certain colour to it. The indicator may thus be a dye or other form of colourant. Suitable fuel dyes useable as indicators include acid/base dyes, fluorescent dyes, diazo dyes and anthraquinone dyes. Suitable red diazo dyes include Solvent Red 19, Solvent Red 24, Solvent Red 26, Solvent Red 161 and Solvent Red 164; suitable green/blue anthraquinone dyes include Solvent Green 33, Solvent Blue 26, Solvent Blue 35, Solvent Blue 79 and Solvent Blue 98; suitable yellow dyes include Solvent Yellow 56 and Solvent Yellow 124. Other suitable indicators useable to mark fuel compositions include those which can be “developed” in order to produce a particular colour or colour change, for example quinizarin and its derivatives (which are initially invisible but can be “developed” using alkali extraction), furfural (again, initially invisible, developed by treatment with aniline acetate), coumarin (initially invisible, detected by alkali extraction which yields a fluorescent complex) and diphenylamine (initially colourless, turns violet/blue on treatment with oxidising acids). Examples of commercially available acid/base indicators include those sold under the trade marks UNIMARK (ex. United Color Manufacturing, USA) and MORTRACE MP (ex. Orgachim/Rohm & Haas).

Such dyes are typically added to fuel compositions in the form of a concentrated solution.

The indicator may mark the fuel composition in another way, for instance with a biochemical tag.

The indicator may produce a spectroscopic response—in particular a change in colour—if subsequently developed in a particular way and/or in response to another subsequent event; its presence in the composition need not therefore be detectable, and/or need not be visible, prior to the developing process or other event. Many acid/base dyes, for example, are initially invisible but can, as described above, be detected by an appropriate developing process which alters the pH of the fuel composition.

Such changes may be used to monitor a property of a fuel composition itself, or as a means of “developing” a sample to detect the presence of the indicator.

An eleventh aspect of the present invention provides a method for preparing a fuel composition, such as a composition according to the tenth aspect, the method involving blending a Fischer-Tropsch derived fuel component with a spectroscopically active indicator and optionally with one or more fuel additives. A non-Fischer-Tropsch derived base fuel may also be included in the composition.

Suitably, the Fischer-Tropsch derived fuel component is incorporated into the composition for one or more of the purposes described above in connection with the first to the tenth aspects of the present invention.

A twelfth aspect of the present invention provides a method for signalling to a user a property of a fuel composition, the method involving including in the composition a Fischer-Tropsch derived fuel component and a spectroscopically active indicator. In this context, a “iuser” includes any person or body involved in the supply, transportation, storage, testing or use of the composition or the handling of the composition for any other purpose.

The method of the twelfth aspect may be used as part of a method for increasing customer loyalty and in turn market share, or of a method for reassuring customers of quality standards, or of a method for detecting counterfeit or illegally traded products, or of a method for quality control of fuel compositions, or of a method for managing the distribution of fuel compositions to users, or of a method for monitoring the areas of use, storage and/or disposal of fuel compositions.

A thirteenth aspect of the present invention provides a method of operating a fuel consuming system, which method involves introducing into the system a fuel composition prepared in accordance with any one of the first to the fourth, the ninth, the eleventh or the twelfth aspects of the present invention, and/or a fuel composition according to the tenth aspect.

The fuel consuming system may in particular be an engine, such as an automotive or aircraft engine, in which case the method may involve introducing the fuel composition into a combustion chamber of the engine. It may be an internal combustion engine, and/or a vehicle which is driven by an internal combustion engine. The engine is preferably a compression ignition (diesel) engine. Such a diesel engine may be of the direct injection type, for example of the rotary pump, in-line pump, unit pump, electronic unit injector or common rail type, or of the indirect injection type. It may be a heavy or a light duty diesel engine.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the present invention may be as described in connection with any of the other aspects.

Other features of the present invention will become apparent from the following examples. Generally speaking, the present invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the present invention are to be understood to be applicable to any other aspect, embodiment or example described herein unless incompatible therewith.

Moreover, unless stated otherwise, any feature disclosed herein may be replaced by an alternative feature serving the same or a similar purpose.

The following non-limiting examples illustrate the properties of fuel compositions prepared in accordance with the present invention.

### EXAMPLE 1

This example illustrates the utility of the present invention in the carrying out of an analytical test on a fuel sample.

A number of diesel fuel compositions were obtained, some conventional petroleum derived fuels and some Fischer-Tropsch derived gas oils. Samples of each fuel were then blended with varying concentrations of a commercially available detergent additive. An indicator useable to determine the presence and approximate concentration of the detergent was then added to the blends, and the colours of the resultant samples were compared. The indicator was of the type which would turn a fuel sample different colours depending on the quantity of detergent present.

For each fuel, five samples were made up as follows: (a) fuel alone, (b) fuel+indicator, (c) fuel+indicator+1 quantity of detergent additive (i.e. detergent additive at its “standard” treat rate), (d) fuel+indicator+2 quantities of detergent additive and (e) fuel+indicator+4 quantities of detergent additive.

The test results were read by eye.

The petroleum derived diesel fuels were initially pale yellow in colour. On addition of the indicator they turned various shades of brown. The samples containing one, two and four quantities of detergent turned brown, greeny-brown and bluey-brown respectively but it was in some cases difficult to distinguish by eye between the three detergent-containing samples for any given fuel.

The Fischer-Tropsch derived gas oils were initially colourless, and on addition of the indicator turned straw-coloured. The samples containing one quantity of detergent turned green on addition of the indicator. The samples containing two quantities of detergent turned blue on addition of the indicator, and those containing four quantities of detergent turned a deep indigo colour. It was relatively straightforward to distinguish by eye between the samples containing different detergent concentrations. It was observed that the colours of the Fischer-Tropsch samples were much more vivid than those of the petroleum derived samples, and the test results correspondingly easier to resolve.

A fuel composition could therefore be prepared containing a blend of a petroleum derived fuel and a Fischer-Tropsch derived fuel component, the presence of the latter serving to improve the response of the overall composition to the indicator, and hence making the composition easier to test for detergent levels.

Other known indicators and fuel markers could be used in a similar way.

### EXAMPLE 2

This example illustrates the benefits of the present invention when incorporating a dye into a fuel composition.

Samples of a range of different fuels, some gasoline and some diesel, were blended with different concentrations of the commercially available blue dye Dyeguard™ Blue 79R (ex. John Hogg Technical Solutions Ltd). The effect of the fuel on the dye colour was observed for each sample. Samples of a Fischer-Tropsch derived gas oil were tested in a similar manner.

The petroleum derived gasoline and diesel fuels were pale yellow or orange in colour. On addition of either 5 or 10 mg/litre of the dye, the pale yellow fuels changed to a murky blue colour whilst the orange fuels turned brown. With 20 mg/litre of dye, the highly coloured orange fuels appeared black in colour. Thus, the petroleum derived fuels, in particular the highly coloured ones, were effectively masking the colour of the blue dye, making it therefore more difficult to detect and/or distinguish in subsequent analytical tests.

The Fischer-Tropsch derived gas oil, in contrast, was colourless and the dye remained a clear blue when incorporated at both 5 and 10 mg/litre. Thus, a Fischer-Tropsch derived fuel may be used in a fuel composition containing a dye such as Dyeguard™ Blue 79R, in order to render the colour of the dyed fuel closer to that of the dye itself, and thus to improve the dye's detectability.

### EXAMPLE 3

Example 2 was repeated but using the commercially available fuel dye Dyeguard™ Green M (ex. John Hogg Technical Solutions Ltd).

Similar results were obtained. The dye initially had a blue/green colour, which was largely unaffected (other than by dilution) by incorporation into the Fischer-Tropsch derived gas oil. When added to the petroleum derived gasoline and diesel fuels, however, the dye appeared as either a murky green colour (in the pale yellow fuels) or an orangey-brown colour (in the more highly coloured fuels). When included at 20 mg/litre in the highly coloured fuels, the ordinarily blue dye appeared to be almost black in colour.

What is claimed is:

1. A method for investigating a property of a fuel composition, the method involving:
(a) adding a spectroscopically active indicator to the composition; and
(b) detecting the presence, absence and/or nature of a spectroscopic response in the composition, on addition of the indicator and/or in response to a subsequent event,
wherein the fuel composition contains a Fischer-Tropsch derived fuel component.

2. A method for investigating a property of a fuel composition which already contains a spectroscopically active indicator, the method comprising detecting the presence, absence and/or nature of a spectroscopic response in the composition in response to a subsequent event, wherein the fuel composition contains a Fischer-Tropsch derived fuel component.

3. The method of claim 1 wherein the spectroscopic response is a visible response.

4. The method of claim 3 wherein the spectroscopic response is a colour change.

5. A fuel composition containing a spectroscopically active indicator and a Fischer-Tropsch derived fuel component.

6. The fuel composition of claim 5 wherein the spectroscopically active indicator is a dye.
